(19) United States
(12) Patent Application Publication
(10) Pub. No.: US 2015/0086951 A1
(43) Pub. Date: Mar. 26, 2015

(54) DEVICE AND METHOD FOR PRIMING A PERSON

(71) Applicant: KONINKLIJKE PHILIPS N.V., EINDHOVEN (NL)

(72) Inventors: Murtaza Bulut, Eindhoven (NL); Tsvetomira Kirova Tsoneva, Eindhoven (NL); Gary Nelson Garcia Molina, Madison, WI (US); Andreea Ioana Sburlea, Zaragoza (ES); Marieke Van Dooren, Arendonk (BE); Mirela Alina Weffers-Albu, Boukoul (NL); Mark Christoph Jaeger, Veldhoven (NL); Raymond Van Ee, Geldrop (NL)

(21) Appl. No.: 14/387,848

(22) PCT Filed: Mar. 29, 2013
(86) PCT No.: PCT/IB2013/052546

§ 371 Date: Sep. 25, 2014

Related U.S. Application Data
(60) Provisional application No. 61/617,086, filed on Mar. 29, 2012.

(51) Int. Cl. G09B 19/00; A61M 21/02

(52) U.S. Cl. CPC G09B 19/00; A61M 21/02; G09B 19/0092; A61M 2021/0083
USPC 434/236

(57) ABSTRACT

The present invention relates to healthy lifestyle management. In particular to a device for priming a person (10), comprising a detector (2) for detecting when the person (10) expresses or should express a predetermined behavior, a stimulus unit (4) for providing the person (10) with a sensory stimulus (14, 16, 21), and a control unit (3) for controlling said stimulus unit (4) to provide the person (10) with a sensory stimulus (14, 16, 21) when it has been detected that the person expresses a predetermined behavior and to provide the same person (10) with the same sensory stimulus (14, 16, 21) when it has been detected that the person (10) expresses or should express said predetermined behavior. A further aspect of the invention relates to a method for priming a person and a computer program for carrying out said method.

### FIELD OF THE INVENTION

The present invention relates to a device, method and computer program for priming a person for carrying out a predetermined desired behavior.

### BACKGROUND OF THE INVENTION

Healthy nutrition and regular exercise are cornerstones in healthy lifestyle management. It is particularly helpful to follow a regular nutrition plan and to avoid any food intake out of regular mealtimes. WO 2010/070645 A1 discloses a method and apparatus for monitoring eating habits. The apparatus comprises a sensor that detects eating events and identifies an eating pattern of the user. An output unit provides the user with feedback about his eating pattern. The feedback can be audible eating instruction, guidance by lighting, a tactile sensation, a release of taste materials and a release of smell materials. The apparatus can be incorporated into various pieces of silverware, such as fork, spoon and knife or alternatively into a plate or as a part of the table. A drawback of the system is that the user is only provided with feedback or guidance about his eating patterns if the user actively engages with the apparatus when eating. Hence, the system only reacts when the user is already eating.

Regular exercise is a second key component in healthy lifestyle management. Although people are aware of this fact, they do not exercise regularly. In addition, adherence to exercise plans is generally low. Even though people schedule regular times for exercise in their calendar, in many cases reminders do not sufficiently stimulate a person to engage in physical activity. Furthermore calendar items are quickly replaced by seemingly more important events. Common for new resolutions is that people decide for an exercise plan, for example start a contract at a gym, but soon afterwards they forget about these plans and return to their sedentary lifestyle.

### SUMMARY OF THE INVENTION

It is an object of the present invention to provide a device and method for priming a person in order to support healthy lifestyle management. It is a further objective of the present invention to proactively support the willingness of a person to engage in a desired behavior.

In a first aspect of the present invention, a device for priming a person is presented that comprises a detector for detecting when the person expresses or should express a predetermined behavior, a stimulus unit for providing the person with a sensory stimulus, and a control unit for controlling said stimulus unit to provide the person with a sensory stimulus when it has been detected that the person expresses a predetermined behavior and to provide the same person with the same sensory stimulus when it has been detected that the person expresses or should express said predetermined behavior.

In a further aspect of the present invention a method for priming a person is presented that comprises detecting when the person expresses or should express a predetermined behavior, providing the person with a sensory stimulus when it has been detected that the person expresses a predetermined behavior, and providing the same person with the same sensory stimulus when it has been detected that the person expresses or should express said predetermined behavior.

In yet another aspect of the present invention, there is provided a computer program which comprises program code means for causing a computer to carry out the steps of the method for priming a person according to the present invention when said computer program is carried out on the computer.

Preferred embodiments of the invention are defined in the dependent claims. It shall be understood that the claimed method, and computer program has similar and/or identical preferred embodiments as the claimed device and as defined in the dependent claims.

Instead of reacting on a user's behavior as known in prior art, the present invention actively assists the user in maintaining a healthy lifestyle. In other words, instead of waiting until the user carries out a specific behavior, the present invention supports the willingness of a person to carry out a specific behavior before carrying out said specific behavior, in other words, priming the user for said specific behavior. In addition to supporting the willingness, the present invention can also initiate the willingness to engage in the primed behavior.

The phenomenon of priming is generally known from cognitive psychology and neuroscience. The psychological phenomenon through which external stimuli activate neural pathways that are related to such stimuli is known as priming. The association between the stimuli and behavior depends on the person's past experience. The device and method according to the present invention can expose the user to one or more stimuli when the user expresses a predetermined behavior to establish a link between a said predetermined behavior and the stimuli and thus build up the person's experience over time. This is also referred to as associative learning.

One can build associations between sensory stimuli and given behaviors through conditioning. A particular form of conditioning termed “classical conditioning” is a reflexive or an automatic type of learning in which a stimulus acquires the capacity to evoke a response that was originally evoked by another stimulus. This phenomenon has first been demonstrated by Pavlov. After associating a sound of a bell with food presentation, he managed to trigger the salivary response of a dog. Salivation is the natural response to food presentation. Even though the dog has not been presented with food yet it can be set in a state ready to eat when hearing the sound of the bell. In this case, the natural stimulus food is replaced by the artificial stimulus bell. In classical conditioning, Pavlov first presents the artificial stimulus for conditioning and afterwards presents the unconditioned stimulus, e.g. meat which naturally makes the dog salivate.

The present invention has identified that it is best to condition a person while the person himself engages in a predetermined behavior by free will. The device according to the present invention detects when the person expresses said predetermined behavior and provides the person with a sensory stimulus. In other words, the present invention monitors the user and detects when he expresses the predetermined desired behavior. Hence, a natural stimulus is not required to elicit a predetermined behavior. After detecting said predetermined behavior, the sensory stimulus for conditioning is provided. In this way, the association between stimulus and predetermined behavior is established. Now that the link between sensory stimulus and the predetermined behavior has been established, the person can be exposed to the sensory stimulus again to increase the likelihood that the person will express said predetermined behavior as a reaction to said sensory stimulus.

In case that the sensory stimulus delivered again while the person is already expressing the predetermined behavior, the purpose of this repeated exposition to the sensory stimulus is to strengthen the conditioning or to maintain it.

In an embodiment of the device according to the present invention, the detector is configured to detect one or more behaviors of a group of behaviors including exercising, doing sports, sleeping, waking up, eating, refraining from eating or working.

The detector is preferably configured to detect when a person expresses or should express said predetermined behavior by measurement of a biometrical or behavioral signal. Examples for biometrical signals include actigraphy, galvanic skin response (GSR), EEG, electrooculogram, electromyography, ECG, video analysis, speech analysis, breathing, sleeping habits, working habits, and so on. Furthermore, the skin conductance, skin temperature, heart rate or heart rate variability, or other additional psycho-physiological parameters can be used as an input to the device. In general, the physiological state determined from biometrical signals, also including an activity monitor, can be evaluated. In general a behavioral signal is derived from monitoring the behavior of the person. In one example, the behavioral signal is derived based on biometrical signals. The behavior in this context relates to actions or feelings. In second example, a video system detects, when the user stays sedentary e.g. in front of a screen all day. In a third example, a behavioral signal is derived from a response of the person to questions, e.g. an inquiry about daily activity. Answers to questions can also provide supplementary information to sensory data.

In a further embodiment, the detector is configured to detect when the person expresses or should express a predetermined behavior automatically from the person's agenda or schedule. A time for going to the gym may be set up as an appointment. If the device does not detect physical activity at the time of the appointment, the person can be primed for exercising. Thus the device according to the invention supports the willingness of the person to go to the gym.

The device for priming a person can further comprise an interface for receiving and/or transmitting information about the person and/or in relation to said predetermined behavior. For example, the device can be supplied with information about the body fitness, age, weight and medical condition of the person. The device can also be configured to communicate with a fitness device so as to verify that the user engaged in the desired physical activity. Information acquired by the device can also be sent to a database for a doctor to have access on the desired behavior of the person.

In a further embodiment, the device for priming a person comprises a sensor for monitoring parameters related to said predetermined behavior. In an exemplary embodiment, this sensor provides further biometrical signals. The sensor can be incorporated into a fitness device and connected to the device for priming a person by the aforementioned interface. Further sensors, such as a door sensor of a fridge, an acceleration sensor, an audio capturing device, or sensors built into a mobile device such as a smart phone, can be added. The sensor can also be configured to monitor food intake or portion control.

In an embodiment, the stimulus unit is configured to provide the person with a group of one or more sensory stimuli out of a set of sensory stimuli including an olfactory stimulus, a stimulus of taste, an audible stimulus, a visual stimulus or a tactile stimulus. In order to support a person in maintaining a healthy personalized meal plan the person can be provided with appetite stimulants such as black pepper, baked bread or vanilla supporting the willingness of a person to eat when it is mealtime according to his nutrition plan. Alternatively, appetite inhibiting scents can be released when it is not the right time for food intake. This can be further combined with a location sensor that releases appetite inhibiting scents when approaching a fast food restaurant with possibly unhealthy food. It should be emphasized that that the various stimuli can be simultaneously presented in order to enhance the effect. Various stimuli can be of the same modality, e.g. a combination of three olfactory stimuli, or of different modality, e.g. two olfactory stimuli in combination with an audible stimulus, such as a jingle, and a video or picture visual stimulus.

In a further aspect of the invention, sensory stimuli are delivered as subliminal stimuli. In other words stimuli are delivered without the user being consciously aware. As an example a weak scent is provided that is not consciously recognized by the user. Alternatively the illumination spectrum of the living room lighting slightly changed so as to resemble the lighting spectrum in a gym. Any stimulus of the group of stimuli can be delivered as a subliminal stimulus.

In yet another embodiment, said stimulus unit is configured to randomly select and provide a sub-group of one or more sensory stimuli from said group of stimuli when it has been detected that the person expresses a predetermined behavior. A random selection of a sub-group of stimuli from a group of stimuli that have been associated with a predetermined behavior can further support the willingness of a person to engage in a specific behavior by preventing habituation to a specific stimulus. For example a sound, smell or picture or any combination thereof can be randomly presented when it has been detected that the person expresses a predetermined behavior. All stimuli from the group are associated with the desired behavior. The willingness of the person to engage in said desired behavior can be randomly supported by any stimulus or sub-group of stimuli out of said group.

In another aspect of the present invention, the control unit is configured to assess the effectiveness of each stimulus in eliciting the predetermined behavior. The response of the person to stimuli can be used to establish a continuously developing and learning system. For example, if it is noted that influence of a certain stimulus or group of stimuli is greater than others, this stimulus can be used more often, and by itself, while stimuli that elicit a weaker response can be used together. In addition, different stimulation algorithms and methodologies, taking into account the past learning about the effectiveness of stimuli can be developed and tailored to the needs of a specific person.

The control unit can also be configured to control said stimulus unit to provide the person with a different sensory stimulus for different behaviors of the same person. For example, a first combination of audible stimulus and olfactory stimulus may be associated with doing sports, an audible stimulus with sleeping and a second combination of audible and olfactory stimulus with dinner.

The device may further comprise a selection unit for allowing the selection of the stimulus for a predetermined behavior. This way the stimulus or group of stimuli can be selected according to user preferences, which ensures better conditioning and priming.

In a further embodiment, the stimulus unit is a fragrance unit comprising a scent container holding a set of fragrances, a scent dispensing unit and a scent control unit for controlling the composition of fragrances in said scent container that are dispensed by said scent dispensing unit.

In a different embodiment of the device according to the present invention, the detector comprises a movement detector in particular an accelerometer, an imaging unit or a motion sensor. This movement detector can monitor the activity level of a person during the day and in case the activity level has been too low issue a stimulus priming the person to engage in exercise.

### BRIEF DESCRIPTION OF THE DRAWINGS

These and other aspects of the invention will be apparent from and elucidated with reference to the embodiments described hereinafter. In the following drawings:

FIG. 1 shows a block diagram of the device for priming a person according to the present invention;

FIG. 2 shows a block diagram of an alternative embodiment of the device for priming a person according to the present invention with additional features;

FIG. 3 shows an embodiment of the present invention during conditioning phase on a fitness device;

FIG. 4 shows an operational circle of the present invention when priming a person for exercise; and

FIG. 5 shows a block diagram of a fragrance unit according to the present invention.

### DETAILED DESCRIPTION OF THE INVENTION

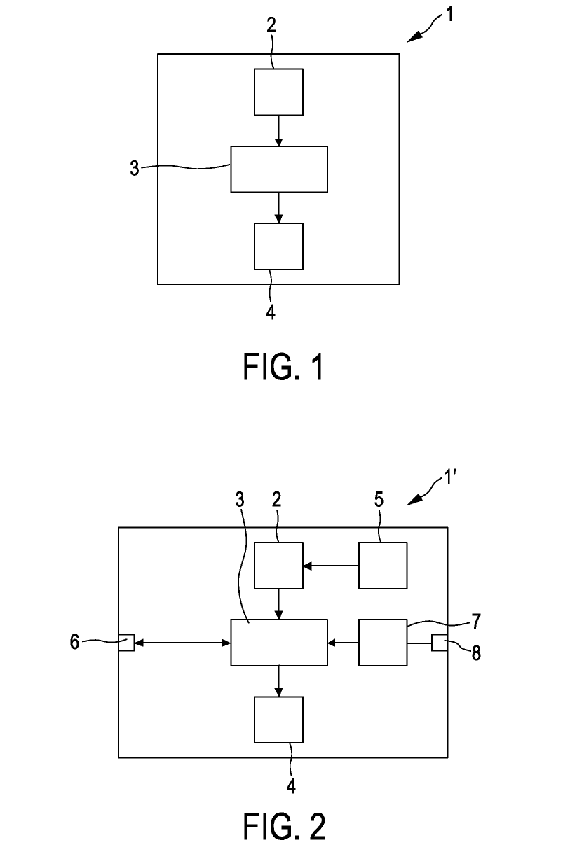

A device for priming a person according to the present invention is illustrated by way of an example in the block diagram shown in FIG. 1. In the illustrated embodiment the device 1 for priming a person comprises a detector 2, a control unit 3, and a stimulus unit 4.

The detector 2 is configured to detect when the person expresses or should express the predetermined behavior. The type of detector 2 depends on the application and is tailored towards the predetermined behavior that is to be detected. The detector can for example be configured to detect when a scheduled event is due. This information is provided to a control unit 3. The control unit 3 in turn controls the stimulus unit 4 to provide the person with a sensory stimulus when it has been detected that the person expresses a predetermined behavior and to provide the same person with the same sensory stimulus when it has been detected that a person expresses or should express said predetermined behavior. The stimulus unit is configured to provide the person with a group of one or more sensory stimuli out of a set of sensory stimuli including an olfactory stimulus, a stimulus of taste, an audible stimulus, a visual stimulus, or a tactile stimulus.

An alternative embodiment of a device 1′ according to the present invention is shown in FIG. 2. In addition to the detector 2, control unit 3 and stimulus unit 4 as shown in FIG. 1, the device 1′ according to this embodiment further comprises one or more of a sensor 5, an interface 6, a selection unit 7, and an interface of the selection unit 8. It should be noted that any of these elements can either be added alone or in various combinations thereof. The sensor 5 in this embodiment is an acceleration sensor. The detector 2 evaluates the signals coming from said acceleration sensor 5 and evaluates how much the person moves during the day. This information in turn is supplied from the detector 2 to the control unit 3.

The device 1′ further comprises an interface 6 for receiving external information. For example the control unit 3 may be coupled to a database that has access to the schedule of a person via the interface 6. Supposedly the acceleration sensor 5 has not sensed sufficient movement of the person during the day then the detector 2 determines that the person should get more active and engage in physical activity. This information is forwarded to the control unit 3. The control unit 3 now queries the database of the person via the interface 6. By this process the control unit 3 may receive the information that the person has scheduled a meeting and that the meeting is going to last about one more hour. Hence, the control unit 3 decides that it is not the right time to stimulate the person for exercise. It is appropriate to wait one more hour until the person will be stimulated for exercise by providing a stimulus via the stimulus unit 4 that has previously been associated with exercise.

In an alternative embodiment the device for priming a person according to the invention as shown in FIG. 2, the sensor 5 is a video camera, for example a webcam mounted on top of the screen where the person is working at. This video data can be evaluated by the detector 2 to determine, whether the person was sitting still in front of the screen all day. Furthermore, the video data may be evaluated to determine whether the person had time for a proper meal or was just eating a little snack in front of the screen. This information is again provided to the control unit 3. The control unit in turn controls the stimulus unit 4 to provide a stimulus for eating according to the desired nutrition plan.

In this embodiment the device for priming a person does not feature an interface 6 but has an additional selection unit 7 and an interface 8 of the selection unit. The interface 8 of the selection unit 7 is a human machine interface that allows the user to select a stimulus that should be applied through the stimulus unit 4 by configuring the control unit 3 through a selection unit 7. Hence the stimulus or group of stimuli is selected according to user preferences, thereby ensuring a higher motivational effect of said stimulus or group of stimuli.

The process of priming a person for a predetermined behavior—in the following example priming for exercise—is a two phase process. An association phase to establish a link between a predetermined behavior and a stimulus is followed by a priming phase to support the willingness of a person to express said predetermined behavior.

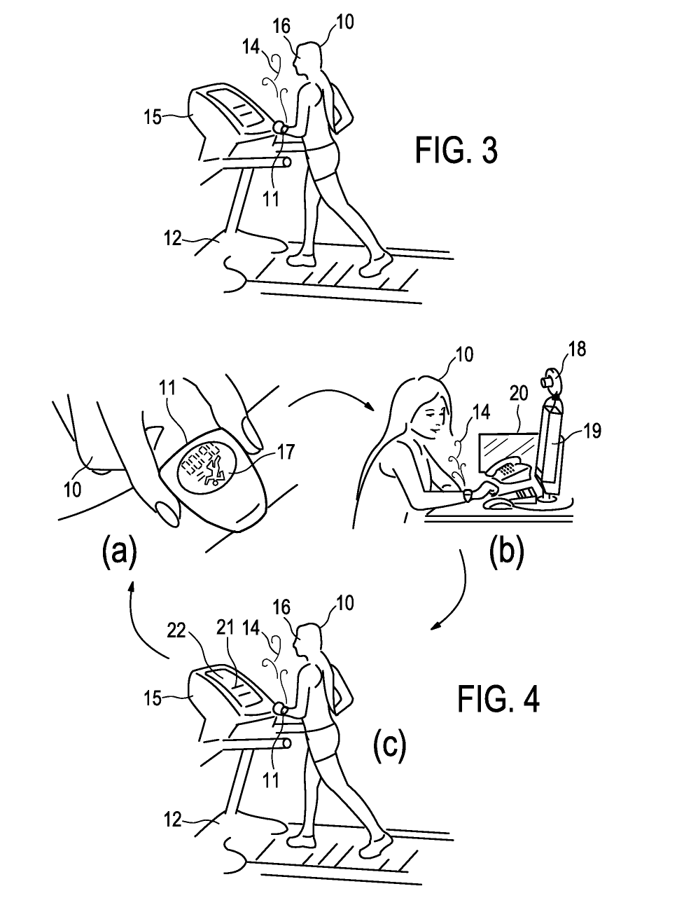

FIG. 3 shows a device 11 for priming a person for exercising during association phase. A person 10 is exercising, for example running. Physiological signals are measured by a sensor 5 as shown in FIG. 2. In this example the physiological signals may include the heart rate provided by a device-internal heart rate sensor. Furthermore, if the exercise is carried out on a fitness device 12, then the fitness device may comprise an additional external sensor 15 connected to the device 11 for priming a person via an external interface 6. When the detector of the device 11 detects a certain arousal level, i.e. when the user is in high arousal state for a certain duration of time, the association phase is triggered. The control unit 3 then controls the stimulus unit 4 to release a stimulus. In this example the stimulus is a combination of an olfactory stimulus 14 and an audible stimulus 16.

For example, when the user has high skin conductance and heart rate level continuously for more than three minutes, it is obvious that he is in highly active state. Observing that the user is maintaining a high activity level for a certain amount of time is an indication that he is motivated and enjoying running. This time of intrinsic motivation when the person expresses a predetermined desired behavior is the right time to release the fragrance and audio stimuli.

If the person is not working out on a fitness device but is running in the field the information from the external sensor 15 of the fitness device can easily be substituted by a different sensor for example an acceleration sensor.

The fragrance stimuli are generally released locally using dispensers that are integrated in the device 11. In the case of local delivery, the fragrance can be directed to the target user. This way other people in the same environment are not influenced. The same holds true for stimuli of different modality. Alternatively, in the case of sparse delivery, the association can be established for multiple people.

In a preferred embodiment the sound is played back by the device 11. However, the device 11 may equally trigger an audio player to play back the audible stimulus. The audible stimulus may be in form of a jingle that is cheerful and memorable. A jingle typically is a short sound of about one to ten, preferably two to five seconds duration. For the case that the user is listening to music during workout, the audible stimulus may be presented either between different songs or at transition points in songs where there are no vocals. In an alternative embodiment the volume of the current song can be lowered and the priming sound can be presented to the user.

Sound and fragrance can be presented at different instants in time or preferably be presented at the same time to achieve the strongest effect by presenting a multi-sensory stimulus. It helps to build up the person's experience and strong association with the said stimuli if the stimuli are presented several times during exercising. Fragrances and sounds can be chosen such that they have energizing effects.

At the end of this association phase the user has built up a link between exercising and the presented stimuli which can now be used for priming the person for exercising as will be explained in the next Figure.

FIG. 4 illustrates the process of priming a person for exercising. In FIG. 4a it is shown that the user sets up a training schedule. In FIG. 4b it is shown that a stimulus or combination of stimuli is presented to the person when the activity is due. In FIG. 4c it is shown that the user engages in a desired activity. A stimulus or combination of stimuli is presented to strengthen the association between activity and stimulus. The steps will now be described in more detail.

In FIG. 4a the device for priming a person is implemented as a device that can be worn around the wrist like a sports watch. The user can program when he/she should express a predetermined behavior, for example running at 6 p.m. Programming can be done via a human-machine-interface 17. Alternatively this information can be provided wired or wireless from an external data source either by the user himself or by medical personnel.

When the time for exercise comes, the user is monitored for a certain amount of time, for example thirty minutes, to determine if he/she indeed follows the plan. The sensor for monitoring can be built into the device 11. Alternatively an external source monitors the person as illustrated in FIG. 4b using the video camera 18 mounted on top of the computer screen 19. When it has been detected that the activity is overdue, the device 11 for priming a person releases one or more of the stimuli 14 that have been associated with that desired action. An audible stimulus can be provided by the device 11 or by an external source such as speakers 20. This stimulus 14 supports the willingness of the person 10 to engage in the predetermined activity by priming the person for said predetermined activity.

The stimuli activate the brain regions that are associated with the physical activity. For example the user recalls his activity (running) and the emotions he/she felt at that time, motor regions for legs and hands become more active. These changes in the user's mental and physical state can also trigger the release of several hormones, such as dopamine. As a result, the user then feels motivated and decides to do his exercises as planned. In consequence, the person 10 is more likely to follow his plan, as shown in FIG. 4c.

Besides scheduling when a predetermined behavior should be expressed, the user may trigger the stimulus on demand. Alternatively the right time for triggering the stimulus can be determined from biometrical signals or activity monitoring.

During the activity, a stimulus or combination of olfactory and audible stimulus can be repeated again to strengthen the association between stimulus and activity.

In an alternative embodiment during repeated exercise as shown in FIG. 4c a different sensory stimulus is applied to the same person for the same activity. The person 10 may be exposed to a visual stimulus 21 presented for example on a display 22 of the fitness device 12 and a second audible stimulus through headphones. A visual stimulus can easily be presented to a user on the computer screen 19. The user now establishes an association with the newly presented stimuli and the same activity. The stimuli previously presented to the person during priming phase and the newly presented stimuli for the same activity now form one group of stimuli associated with said activity.

In order to prevent habituation the stimulus unit may randomly select any sub-group of one or more sensory stimuli from said group of stimuli associated with said predetermined behavior.

Furthermore, the control unit assesses the effectiveness of each stimulus, group or sub-group of stimuli in eliciting the predetermined behavior. For example one person is more responsive to audible and olfactory stimuli, while another person is stimulated best with visual stimuli.

Of course, it is also possible to configure the stimulus unit to provide the person with a different sensory stimulus or group of different sensory stimuli for different behaviors of the same person.

In a further embodiment the person can be primed for recovery after exercising. Similar to the embodiment mentioned before, the person is now provided with a different sensory stimulus when the detector 5 has detected that the person is in a relaxed quiet state, for example by evaluating a regular breathing frequency and a low heart rate with the sensor 5 that is configured to detect biometrical signals. After exercising the user can be exposed to said stimulus that is associated with relaxation to help recover faster.

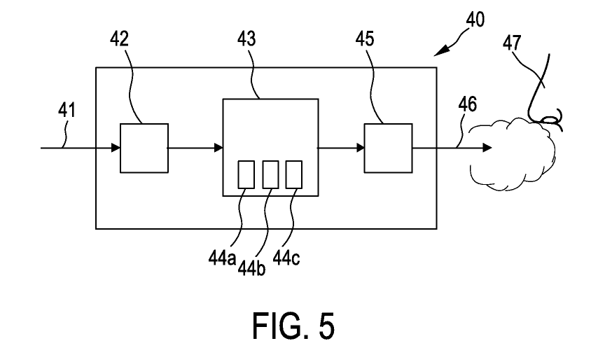

FIG. 5 shows a specific embodiment of a stimulus unit as a fragrance unit 40. The fragrance unit 40 receives a control signal 41 from the control unit. The signal is processed by the scent control unit 42. The scent control unit 42 in turn controls the release of different fragrances 44a, 44b, 44c that are contained in a scent container 43. The scent control unit may select an individual fragrance or any combination thereof. The selected fragrances are then released through the scent dispensing unit 45. An olfactory stimulus 46 is released from the fragrance unit 40 to be perceived by the user 47.

The present invention is not limited to priming a person for specific actions but can also assist in priming a person for specific feelings. Referring again to FIG. 2, the sensor 5 can be an audio-capturing device. The detector 2 evaluates a signal coming from said audio sensor 5. In one embodiment the detector 2 detects laughter in said audio stream. Laughter is an indicator for positive feelings. Hence, a stimulus can be applied through the stimulus unit 4 by control unit 3 in case laughter has been detected in order to associate said positive feelings with said stimulus. When it is desired that the person should express said positive feelings, the user can be provided with said stimulus associated with the positive feelings to lift the mood of the person.

In an embodiment, the fragrance unit 40 dispenses satiety-priming fragrances to diminish the appetite of the user. The user can activate or de-activate the fragrance unit 40 depending on her/his preferences. The schedule for dispensing the appetite suppressant scents is preferably defined by taking into account the craving frequency, which is e.g. known from M. Pelchat, “Food Cravings in Young and Elderly Adults,” Appetite, vol. 28, 1997. Consequently, fragrance dispensing is done more frequently in the early evening.

The scent container 43 in such an embodiment has appetite suppressing scents such as citric scents (e.g. lemon, grapefruit), peppermint, or cinnamon (see e.g. http://www.ehow.com/list_{—}6883981_fragrances-decrease-appetite.html). The type of fragrance to be dispensed at a particular moment is randomly selected out of the set of fragrances in the scent container. Random selection prevents habituation.

In another embodiment the scent control unit 42 includes a craving control program as e.g. described above for the previous embodiment. In addition, the scent control unit 42 primes the appetite of the user at the times specified by the user's nutrition program using appetite-stimulant scents. The scent container 43 in such an embodiment does not only contain the appetite-suppressant scents but also appetite-stimulant scents such as black pepper, baked bread, or vanilla. Extra-sensors can be used in this embodiment to monitor food intake. In this case portion control can be achieved by notifying the user when the consumed food exceeds planned limits.

It is also an option to request the user to notify of her/his meals using a mobile phone application. Various mobile-phone applications exist to help the user in getting awareness of her/his eating habits.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A device for priming a person, comprising:
a detector for detecting when the person expresses or should express a predetermined behavior,
a stimulus unit for providing the person with a sensory stimulus, and
a control unit for controlling said stimulus unit to provide the person with a sensory stimulus when it has been detected that the person expresses a predetermined behavior and to provide the same person with the same sensory stimulus when it has been detected that the person should express said predetermined behavior.

2. The device according to claim 1, wherein said detector is configured to detect one or more behaviors of a group of behaviors including exercising, doing sports, sleeping, waking up, eating, refraining from eating, or working.

3. The device according to claim 1, wherein said detector is configured to detect when the person expresses or should express said predetermined behavior by measurement of a biometrical or behavioral signal.

4. The device according to claim 1, wherein said detector is configured to detect when the person expresses or should express said predetermined behavior automatically from the person's agenda or schedule.

5. The device according to claim 1, further comprising an interface for receiving and/or transmitting information about the person and/or in relation to said predetermined behavior.

6. The device according to claim 1, further comprising a sensor for monitoring parameters related to said predetermined behavior.

7. The device according to claim 1, wherein said stimulus unit is configured to providing the person with a group of one or more sensory stimuli out of a set of sensory stimuli including an olfactory stimulus, a stimulus of taste, an audible stimulus, a visual stimulus or a tactile stimulus.

8. The device according to claim 7, wherein said stimulus unit is configured to randomly select and provide a sub-group of one or more sensory stimuli from said group of stimuli when it has been detected that the person expresses a predetermined behavior.

9. The device according to claim 1, wherein said control unit is configured to assess the effectiveness of each stimulus, group or sub-group of stimuli in eliciting the predetermined behavior.

10. The device according to claim 1, wherein said control unit is configured to control said stimulus unit to provide the person with a different sensory stimulus for different behaviors of the same person.

11. The device according to claim 1, further comprising a selection unit allowing the selection of a stimulus for a predetermined behavior.

12. The device according to claim 1, wherein said stimulus unit is a fragrance unit comprising:
a scent container for holding a set of fragrances,
a scent dispensing unit, and
a scent control unit for controlling the composition of fragrances in said scent container that are dispensed by said scent dispensing unit.

13. The device according to claim 1, wherein said detector comprises a movement detector, in particular an accelerometer, an imaging unit or a motion sensor.

14. A method for priming a person, comprising:
detecting when the person expresses or should express a predetermined behavior,
providing the person with a sensory stimulus when it has been detected that the person expresses a predetermined behavior, and
providing the same person with the same sensory stimulus when it has been detected that the person should express said predetermined behavior.

15. Computer program comprising program code means for causing a computer to carry out the steps of the method as claimed in claim 14 when said computer program is carried out on the computer.
